(19) United States
(12) Patent Application Publication
(10) Pub. No.: US 2007/0249669 A1
(43) Pub. Date: Oct. 25, 2007

(54) CRYSTALLINE 2-AMINO-3-CYANOQUINOLINE DERIVATIVES, PROCESS OF THEIR PREPARATION AND PHARMACEUTICAL COMPOSITIONS CONTAINING THEM

(75) Inventors: Zoltan Finta, Budapest (HU); Istvan Hermecz, Budapest (HU); Gergely Heja, Szentendre (HU); Agnes Horvath, Budapest (HU); Gyulane Kiss, Budapest (HU); Miklos Morvai, Mogyorod (HU); Benjamin Podanyi, Dunakeszi (HU); Judit Sipos, Budapest (HU); Anna Szabo, Budapest (HU); Arpadne Vasvari, Budapest (HU); Erika Varkonyine Schlovicsko, Budapest (HU)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: sanofi-aventis, Paris (FR)

(21) Appl. No.: 11/550,598

(22) Filed: Oct. 18, 2006

Related U.S. Application Data
(63) Continuation of application No. PCT/HU05/00036, filed on Apr. 14, 2005.

(30) Foreign Application Priority Data
Apr. 19, 2004 (HU) P0400812

(51) Int. Cl. C07D 215/38

(52) U.S. Cl. 514/313; 546/159

(57) ABSTRACT

The invention relates to the crystalline and amorphous forms of the desmotrope of general formula IB and its salts and solvates, and their preparation.

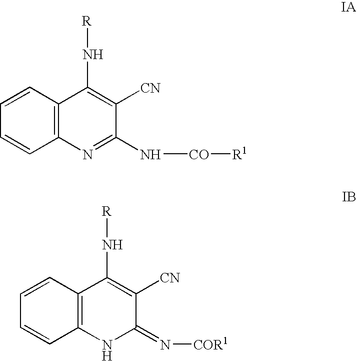

This application is a Continuation of International Patent Application No. PCT/HU2005/000036, filed 14 Apr. 2005, and claims the benefit of Hungarian Patent Application P 0400812, filed 19 Apr. 2004; both of which applications are hereby incorporated by reference in their entirety.

The subjects of the present invention are: process for the preparation of 2 different crystalline forms of 2-amino-3-cyanoquinoline derivatives; crystalline forms A and B; and pharmaceutical preparations containing them.

Patent application WO 02/096879 describes 2-amino-3-cyanoquinoline derivatives of the general formula (1) and their solvates and isomers.

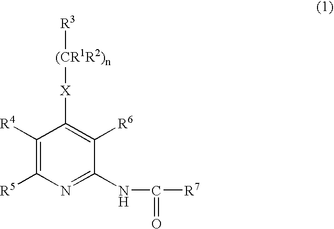

In general formula (I) the substituents have the following meanings:

R¹ represents hydrogen atom or C₁₋₄ straight or branched alkyl group;

R² represents hydrogen atom or C₁₋₄ straight or branched alkyl group;

R³ represents hydrogen atom; C₁₋₄ straight or branched alkyl group; phenyl-, thienyl-, or furyl group—optionally substituted with one or more C₁₋₄ straight or branched alkyl group, C₁₋₄ straight or branched alkoxy group, or halogen atom; a 5 or 6-membered heteroaromatic ring containing one, two or three nitrogen atoms, or one nitrogen atom and one oxygen atom, or one nitrogen and one sulphur atom—optionally substituted with one or more C₁₋₄ straight or branched alkyl group, C₁₋₄ straight or branched alkoxy group, or halogen atom;

R⁴ and R⁵ represent hydrogen atom; or may together form a 1,3-butadienyl group—optionally substituted with a methylenedioxy group or with one or more C₁₋₄ straight or branched alkyl group, C₁₋₄ straight or branched alkoxy group, hydroxyl group or halogen atom;

R⁶ represents hydrogen atom, cyano group, aminocarbonyl group, C₁₋₄ alkoxycarbonyl group or carboxy group;

R⁷ represents hydrogen atom; C₁₋₄ straight or branched alkyl group; phenylbenzyl-, thienyl- or furyl group—optionally substituted with one or more C₁₋₄ straight or branched alkoxy group, hydroxyl group, trifluoromethyl group, cyano group, or halogen atom; or a 5 or 6-membered heteroaromatic ring containing one, two or three nitrogen atoms, or one nitrogen and one oxygen atom, or one nitrogen and one sulphur atom—optionally substituted with one or more C₁₋₄ straight or branched alkyl group, C₁₋₄ straight or branched alkoxy group, or halogen atom;

X stands for —CH₂—, —NH—, or —NR⁸ group, sulphur or oxygen atom, sulpho- or sulphoxy group—wherein R⁸ represents C₁₋₄ straight or branched alkyl group, or C₃₋₆ cycloalkyl group;

n has the value of zero, 1 or 2.

According to the process described in patent application WO 02/096879 the 2-amino-3-cyanoquinoline derivatives of the general formula (I) are prepared by acylation of the 4-substituted 2-amino-3-cyanoquinoline derivative, followed by selective hydrolysis of the resulting intermediate.

NMR spectra of the compounds the general formula (I) in solution reveal the presence of 2 tautomeric forms:

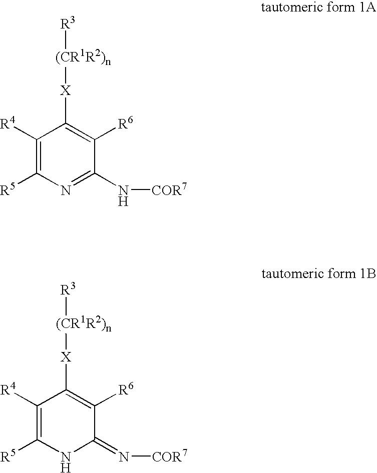

(In the formulae the substituents have the same meanings as defined above for general formula (1).)

In solution the tautomeric forms 1A and 1B are in equilibrium, defined by the solvent and the temperature.

Patent application WO 02/096879 renders tautomeric form 1A to the compounds—which is the dominating form in solution.

Surprisingly we have found, that with the compounds of general formula (I)—falling under the scope of general formula (1), 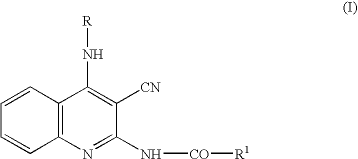
wherein

R represents benzyl-, 2-(thienyl)methyl- or 2-(furyl)methyl group and

R¹ represents phenyl group or furyl group substituted with C₁₋₄ alkoxy group or halogen atom

the two tautomeric forms may be isolated separately in solid state, and the resulting desmotropic forms in crystalline state may be stored practically without time limit. Depending on the conditions of the crystallisation, form IA or form IB may be obtained, and these forms are to consider as desmotropes.

On the basis of solid state IR and ¹⁵N and ¹³C NMR studies, the following general structures were assigned to desmotrope IA and desmotrope IB, respectively:

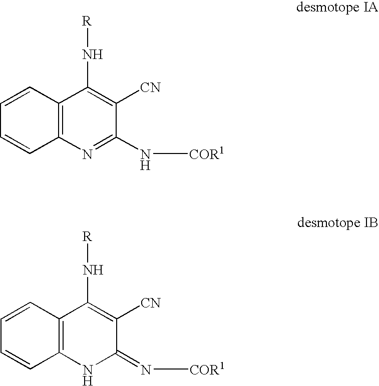

(In the formulae the substituents have the same meanings as defined above for the general formula (I).)

Desmotrope IA is the solid form of tautomer (1A), whereas desmotrope IB is the solid form of tautomer (1B).

Comparing the spectral characteristics of the desmoptropes, we can state that in the IR spectra the biggest differences can be found in the positions of the characteristic amide C═O absorption bands, which appear in desmotrope A around 1670 cm⁻¹, whereas in desmotrope B around 1620 cm⁻¹.

In the solid state ¹³C NMR spectra significant differences between the respective chemical shifts of the two desmotropes can be found for the carbon atoms in positions 2, 3, and 8a of the quinoline ring, and for the carbonyl carbon atom; whereas in the ¹⁵N NMR spectra for the quinoline nitrogen and for the N atom attached to the carbonyl group.

Desmotropes IA and IB can be transformed into one and other by recrystallisation and they may contain solvents in the form of solvates.

In the light of the above, the subject of our invention is a process for the preparation of the crystalline or amorphous forms of desmotropes IA and IB 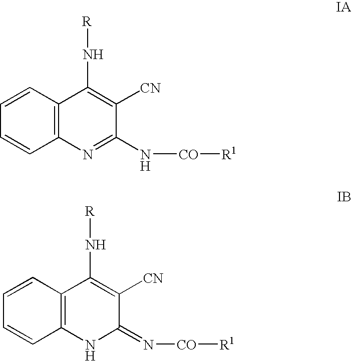
where in the formulae
   R represents benzyl-, 2-(thienyl)methyl- or 2-(furyl)methyl group and
   R¹ represents phenyl or furyl group substituted with C1-4 alkoxy group or halogen atom
   by acylation of the appropriate 4-substituted-2-amino-3-cyanoquinoline derivative and selective hydrolysis of the resulting intermediate, characterized in that,
a.) to prepare desmotrope IA, the product is crystallized from isobutanol
b.) to prepare desmotrope IB, the acylation reaction is carried out in a polar solvent and—if desired the product is crystallized from a polar solvent, or
c.) to prepare desmotrope IB, desmotrope IA or the mixture of any ratio of the desmotropes IA and IB is crystallized from a polar, apolar or dipolar aprotic solvent—in a given case after seeding with the crystals of desmotrope IB.

In variant b.) the reaction is preferably carried out in 2-butanone, acetonitrile or ethanol.

In variant c.) the desmotrope IA or the mixture of any ratio of the desmotropes IA and IB is preferably crystallized from a polar, apolar or dipolar aprotic solvent. As for solvent preferably chloroform, 2-butanone or methanol are used.

Characteristics of the desmotropes IA and IB of the individual compounds are given in the examples.

Further subjects of the invention are the desmotropes IB and their salts and solvates.

Biological activities of the desmotropes IA and IB according to the invention are identical, but physical characteristics, among them solubilities, permeabilities, etc. may be different, thus in pharmaceutical preparations the most appropriate desmotrope for the given pharmaceutical form may be used.

Preparation and description of the desmotropes IA and IB according to the invention and results of the structural assignments are given in the examples below, without limiting the invention to the examples.

### EXAMPLES

### Example 1

### 4-Methoxy-N-(4-benzylamino-3-cyano-quinolin-2-yl)benzamide

In general formula (IA) R¹ represents benzyl group, R² represents 4-methoxyphenyl group.

To the solution of 1 g of 3-methoxy-N-(3-methoxybenzoyl)-N-(4-benzylamino-3-cyanoquinolin-2-yl)benzamide in 16 ml of acetonitrile, 4 ml of 1N methanolic potassium hydroxide solution is added. The reaction mixture is heated under reflux for 3 minutes; 0.6 ml of acetic acid glacial is added to it, the mixture is neutralized with 10 ml of 1M sodium hydrogencarbonate solution, the resulting crystals are dissolved hot in 15 ml of 1-butanol and filtered. The solution is slowly (0.2° C./min) cooled to room temperature.

0.65 g of the title compound is obtained, m.p.: 188-191° C.

IR: ν CN: 2220; ν CO: 1670 cm⁻¹

SsNMR ¹³C (ppm): C (2) 153.9; C (3) 87.4; C (8a) 146.6; CO 167.1. ¹⁵N (ppm): N (1) −136.2; amide-N −244.3; cyano-N −105.1; benzylamino-N −297.6.

### Example 2

### N-[(2Z)-4-(benzylamino)-3-cyanoquinolin-2(1H)-ylidene]-4-methoxybenzamide

In general formula (IB) R represents benzyl group, R¹ represents 4-methoxyphenyl group.

To the solution of 1.2 g of 4-methoxy-N-(4-methoxybenzoyl)-N-(4-benzylamino-3-cyanoquinolin-2-yl)benzamide in 30 ml of 2-butanone, 5 ml of 1N sodium ethylate solution in ethanol is added. The reaction mixture is heated under reflux for 4 minutes; 0.7 ml of acetic acid glacial is added to it, the mixture is neutralized with 12.5 ml of 1M sodium hydrogencarbonate solution, the resulting yellow crystalline material is filtered off.

0.45 g (49%) of the title compound is obtained, m.p.: 178-180° C.

IR: ν CN: 2205; ν CO: 1620 cm⁻¹

SsNMR ¹³C (ppm): C (2) 158.2 C; C (3) 81.2; C (8a) 135.4; CO 176.7. ¹⁵N (ppm): N (1) −240.2; imino-N −185.4; cyano-N −113.8 benzylamino-N −280.9;

### Example 3

The compound of Example 2 may also be prepared in the following way:

2.0 g of N-[4-(benzylamino)-3-cyano-2-quinolinyl]-4-methoxybenzamide is dissolved in 30 ml of chloroform at reflux temperature. The solution is filtered hot, slowly (−0.2° C./min) cooled to room temperature and allowed to stay at room temperature for 1 night. Next day the solid material is filtered off, dried in vacuum at 40° C.

0.98 g (49%) of product is obtained.

Mp: 180-182° C.

IR: ν CN: 2205; ν CO: 1620 cm⁻¹

### Example 4

The compound of Example 2 may also be prepared in the following way:

The suspension of 2.0 g of N-[4-(benzylamino)-3-cyano-2-quinolinyl]-4-methoxybenzamide in 50 ml of 2-butanone is stirred at room temperature for 10 days. The product is filtered off, dried in vacuum at 60° C. for 3 hours.

0.74 g (37%) of product is obtained.

Mp: 180-182° C.

IR: ν CN: 2214; ν CO: 1620 cm⁻¹

### Example 5

The 2:1 ratio solvate with ethanol of the compound of Example 2 may also be prepared in the following way:

5.0 g of N-[(2Z)-4-(benzylamino)-3-cyanoquinolin-2(1H)-ylidene]-4-methoxybenzamide is dissolved at reflux temperature in 210 ml of 96% ethanol and filtered hot. The solution is slowly (−0.2° C./min) cooled to room temperature and allowed to stay at room temperature for 1 night. Next day the resulted precipitate is filtered off, washed with 3 ml of 96% ethanol of ambient temperature and dried in vacuum at 60° C.

4.39 g (83.14%) of product is obtained.

Mp: 110-122° C.

IR: ν CN: 2206; ν CO: 1663 cm⁻¹

### Example 6

The 2:1 ratio solvate with 1-propanol of the compound of Example 2 is prepared in the following way:

1.0 g of N-[(2Z)-4-(benzylamino)-3-cyanoquinolin-2(1H)-ylidene]-4-methoxybenzamide is dissolved at reflux temperature in 33 ml of 1-propanol and filtered hot. The solution is slowly (−0.2° C./min) cooled to room temperature and the resulted precipitate is filtered off, washed with 5 ml of 1-propanol of ambient temperature and dried in vacuum at 60° C. for 3 hours.

0.91 g (84.8%) of product is obtained.

Mp: 111-134° C.

IR: ν CN: 2210; ν CO: 1664 cm⁻¹

### Example 7

The 2:1 ratio solvate with 2-propanol of the compound of Example 2 is prepared in the following way:

0.7 g of N-[(2Z)-4-(benzylamino)-3-cyanoquinolin-2(1H)-ylidene]-4-methoxybenzamide is dissolved at reflux temperature in 70 ml of 2-propanol and filtered hot. The solution is allowed to cool to room temperature and the resulted precipitate is filtered off, washed with 2 ml of 1-propanol of ambient temperature and dried in vacuum at 60° C. for 3 hours.

0.52 g (69.3%) of product is obtained.

Mp: 111-134° C.

IR: ν CN: 2210; ν CO: 1664 cm⁻¹

### Example 8

The 1:1 ratio solvate with 2-propanol of the compound of Example 2 is prepared in the following way:

30 mg of N-[(2Z)-4-(benzylamino)-3-cyanoquinolin-2(1H)-ylidene]-4-methoxybenzamide is dissolved at reflux temperature in 2.65 ml of 2-propanol and filtered hot. The solution is immediately placed into a bath of crushed ice, cooled by shock-cooling to 0° C. and kept at −5° C. overnight. The resulted precipitate is filtered off, washed with 0.5 ml of 1-propanol and dried in vacuum at 60° C. for 3 hours.

21 mg (61.0%) of product is obtained.

Mp: 110-128° C.

IR: ν CN: 2215; ν CO: 1620 cm⁻¹

### Example 9

The 2:1 ratio solvate with 2-methyl-1-propanol of the compound of Example 2 is prepared in the following way:

100 mg of N-[(2Z)-4-(benzylamino)-3-cyanoquinolin-2(1H)-ylidene]-4-methoxybenzamide is dissolved at reflux temperature in 2.8 ml of 2-methyl-1-propanol and filtered hot. The solution is allowed to cool to room temperature, the resulted precipitate is filtered off, washed with 2 ml of 2-methyl-1-propanol of ambient temperature and dried in vacuum at 60° C. for 3 hours.

67.3 mg (61.7%) of product is obtained.

Mp: 110-131° C.

IR: ν CN: 2206; ν CO: 1667 cm⁻¹

### Example 10

The 1:1 ratio solvate with N-methylpyrrolidone of the compound of Example 2 is prepared in the following way:

200 mg of N-[(2Z)-4-(benzylamino)-3-cyanoquinolin-2(1H)-ylidene]-4-methoxybenzamide is dissolved at 90° C. in 0.5 ml of N-methylpyrrolidone and filtered hot. The solution is slowly (−0.2° C./min) cooled to room temperature, the resulted precipitate is filtered off and dried in vacuum at 60° C. for 3 hours.

113.5 mg (45.7%) of product is obtained.

Mp: 105-116° C.

IR: ν CN: 2212; ν CO: 1664 cm⁻¹

### Example 11

The amorphous form of the compound of Example 2 is prepared in the following way:

1.5 g of N-[(2Z)-4-(benzylamino)-3-cyanoquinolin-2(1H)-ylidene]-4-methoxybenzamide is dissolved at room temperature in 300 ml of dichloromethane and the solution is filtered. From a water-bath of 35° C. the clear filtrate is quickly evaporated to dryness in vacuum.

1.41 g (94%) of product is obtained.

Mp: 88-103° C.

IR: ν CN: 2210; ν CO: 1622 cm⁻¹

### Example 12

### N-[(2Z)-4-(benzylamino)-3-cyanoquinolin-2(1H)ylidene]-4-fluorobenzamide

In general formula (IB) R represents benzyl group, R¹ represents 4-fluorophenyl group.

To the solution of 0.26 g of 4-fluoro-N-(4-fluorobenzoyl)-N-(4-benzylamino-3-cyanoquinolin-2-yl)benzamide in 6 ml ethanol, 0.92 ml of 1N potassium hydroxide solution in methanol is added. The reaction mixture is heated under reflux for 5 minutes; 0.14 ml of acetic acid glacial is added to it, the mixture is neutralized with 2.5 ml of 1M sodium hydrogencarbonate solution, the resulting yellow crystalline material is filtered off.

0.12 g of the title compound is obtained, m.p.: 190-193° C.

IR: ν CN: 2214; ν CO: 1620 cm⁻¹

### Example 13

### N-[(2Z)-4-(thienylmethylamino)-3-cyanoquinolin-2(1H)-ylidene]-4-methoxybenzamide

In general formula (IB) R represents thienylmethyl group, R¹ represents 4-methoxyphenyl group.

By selective hydrolysis of the 4-methoxy-N-(4-methoxybenzoyl)-N-(4-thienylmethylamino-3-cyanoquinolin-2-yl)benzamide, by a process analogous to Example 12, the title compound was obtained (64%), mp.: 169-171° C.

IR: ν CN: 2203; ν CO: 1622 cm⁻¹

### Example 14

The compound of Example 13 may also be obtained in the following way:

1.0 g N-[4-(thienylmethylamino)-3-cyano-2-quinolinyl]-4-methoxybenzamide is dissolved at reflux temperature in 55 ml of methanol and filtered hot. The solution is slowly (−0.2° C./min) cooled to room temperature and allowed to stay at room temperature overnight. The resulting precipitate is filtered off and dried in vacuum at 40° C.

0.88 g (88%) product is obtained.

Mp: 168-170° C.

IR: ν CN: 2203; ν CO: 1622 cm⁻¹

### Example 15

The 1:1 ratio hydrate of the compound of Example 13 is prepared in the following way:

30 mg N-[(2Z)-4-(thienylmethylamino)-3-cyanoquinolin-2(1H)-ylidene]-4-methoxybenzamide is dissolved at reflux temperature in 3 ml of 90% ethanol and filtered hot. The solution is slowly (−0.2° C./min) cooled to room temperature and allowed to stay overnight at room temperature. The product is filtered off and dried in vacuum at 40° C.

21 mg (67.1%) of product is obtained.

Mp: 110° C. (shrinking) 170-171° C.

IR: ν CN: 2207; ν CO: 1622 cm⁻¹

### Example 16

### N-[(2Z)-4-(furylmethylamino)-3-cyanoquinolin-2(1H)-yilidene]-4-methoxybenzamide

In general formula (IB) R represents furylmethyl group, R¹ represents 4-methoxyphenyl group.

By selective hydrolysis of the 4-methoxy-N-(4-methoxybenzoyl)-N-(4-furylmethylamino-3-cyanoquinolin-2-yl)benzamide, by a process analogous to Example 12, the title compound was obtained (71%), mp.: 114-116° C.

IR: ν CN: 2209; ν CO: 1621 cm⁻¹

### Example 17

The compound of Example 16 may also be obtained in the following way:

30 mg of N-[4-(furylmethylamino)-3-cyano-2-quinolinyl]-4-methoxybenzamide is dissolved at reflux temperature in 0.93 ml of 2-butanone and filtered hot. The solution is slowly (−0.2° C./min) cooled to room temperature and allowed to stay at room temperature overnight. The resulting precipitate is filtered off and dried in vacuum at 40° C.

0.18 mg (60%) product is obtained.

Mp: 110-115° C.

IR: ν CN: 2209; ν CO: 1621 cm⁻¹

### Example 18

### N-[(2Z)-4-(furylmethylamino)-3-cyanoquinolin-2(1H)-ylidene]-2-furyl carboxamide

In general formula (IB) R represents furylmethyl group, R¹ represents 2-furyl group.

By selective hydrolysis of the N-(2-furancarbonyl)-N-(4-(2-furylmethylamino)-3-cyanoquinolin-2-yl)furan-2-carboxamide, by a process analogous to Example 12, the title compound was obtained (88%), mp.: 188-191° C.

IR: ν CN: 2212; ν CO: 1621 cm⁻¹

Melting points were determined by the capillary method in Büchi 535 apparatus and in Boetius PHMK 05 apparatus following the melting by microscopy.

IR spectra were recorded with Bruker IFS-28FT-IR instrument using KBr pellets in the range of 4000-400 cm⁻¹.

Solid state NMR studies were performed using Bruker DRX-500 spectrometer, according to parameters:

| | |
|---|---|
| Head: | 4 mm standard MAS head |

Nucleus ¹³C

| | |
|---|---|
| Spectrum window | 34.0 kHz |
| Detection time | 60 ms |
| Relaxation time | 15 s |
| Number of data points | 4096 zero-filled to 8k |

Nucleus: ¹⁵N

| | |
|---|---|
| Spectrum window | 30.3 kHz |
| Detection time | 51 ms |
| Relaxation time | 15 s |
| Number of data points | 3072 zero-filled to 8k |
| Spinning speed | 12.0 kHz |
| Temperature | ambient (298K) |

We claim:

1. A crystalline form of the desmotrope of general formula IB 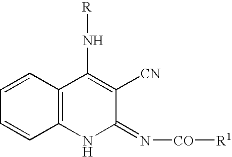
and its salts and solvates,
wherein
R represents benzyl-, 2-(thienyl)methyl- or 2-(furyl)methyl-group and
R¹ represents phenyl or furyl group substituted with C₁₋₄ alkoxy group or halogen atom.

2. An amorphous form of the desmotrope of general formula IB 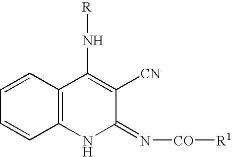
and its salts and solvates,
wherein
R represents benzyl-, 2-(thienyl)methyl- or 2-(furyl)methyl-group and
R¹ represents phenyl or furyl group substituted with C₁₋₄ alkoxy group or halogen atom.

3. The compound according to claim 1, selected from:
N-[(2Z)-4-(benzylamino)-3-cyanoquinolin-2(1H)-ylidene]-4-fluorobenzamide;
N-[(2Z)-4-(benzylamino)-3-cyanoquinolin-2(1H)-ylidene]-4-methoxybenzamide;
N-[(2Z)-4-(thienylmethylamino)-3-cyanoquinolin-2(1H)-ylidene]-4-methoxybenzamide;
N-[(2Z)-4-(furylmethylamino)-3-cyanoquinolin-2(1H)-ylidene]-4-methoxybenzamide; and
N-[(2Z)-4-(furylmethylamino)-3-cyanoquinolin-2(1H)-ylidene]-2-furylcarboxamide;
and it's salts and solvates.

4. The compound according to claim 2, selected from:
N-[(2Z)-4-(benzylamino)-3-cyanoquinolin-2(1H)-ylidene]-4-fluorobenzamide;
N-[(2Z)-4-(benzylamino)-3-cyanoquinolin-2(1H)-ylidene]-4-methoxybenzamide;
N-[(2Z)-4-(thienylmethylamino)-3-cyanoquinolin-2(1H)-ylidene]-4-methoxybenzamide;
N-[(2Z)-4-(furylmethylamino)-3-cyanoquinolin-2(1H)-ylidene]-4-methoxybenzamide; and
N-[(2Z)-4-(furylmethylamino)-3-cyanoquinolin-2(1H)-ylidene]-2-furylcarboxamide;
and it's salts and solvates.

5. A process for the preparation of a crystalline form of desmotrope IA: 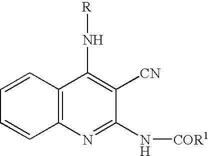
wherein
R represents benzyl-, 2-(thienyl)methyl- or 2-(furyl)methyl-group and
R¹ represents phenyl or furyl group substituted with C₁₋₄ alkoxy group or halogen atom,
by acylation of the appropriate 4-substituted-2-amino-3-cyanoquinoline derivative and selective hydrolysis of the resulting intermediate, comprising:
crystallizing the product from isobutanol.

6. A process for the preparation of a crystalline or amorphous form of desmotrope IB: 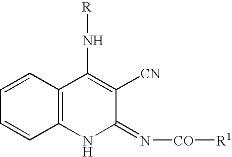
wherein
R represents benzyl-, 2-(thienyl)methyl- or 2-(furyl)methyl-group and
R¹ represents phenyl or furyl group substituted with C₁₋₄ alkoxy group or halogen atom,
by acylation of the appropriate 4-substituted-2-amino-3-cyanoquinoline derivative and selective hydrolysis of the resulting intermediate, comprising:
acylating the appropriate 4-substituted-2-amino-3-cyanoquinoline derivative in a polar solvent and optionally crystallizing from a polar solvent.

7. A process for the preparation of a crystalline form of desmotrope IB: 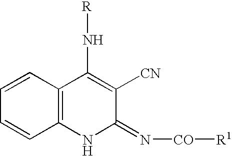
wherein
R represents benzyl-, 2-(thienyl)methyl- or 2-(furyl)methyl-group and
R¹ represents phenyl or furyl group substituted with C₁₋₄ alkoxy group or halogen atom,
by acylation of the appropriate 4-substituted-2-amino-3-cyanoquinoline derivative and selective hydrolysis of the resulting intermediate, comprising:
crystallizing desmotrope IA or a mixture of any ratio of the desmotropes IA and IB from a polar, apolar or dipolar aprotic solvent, in a given case after seeding with crystals of desmotrope IB.

8. A process according to claim 6 wherein the solvent is selected from 2-butanone, acetonitrile or ethanol.

9. A process according to claim 7 wherein the solvent is selected from chloroform, 2-butanone, or methanol.

10. A pharmaceutical composition comprising the crystalline form of the desmotrope of formula IB according to claim 1.

11. A pharmaceutical composition comprising the amorphous form of the desmotrope of formula IB according to claim 2.

12. A pharmaceutical composition comprising the compound according to claim 3.

13. A pharmaceutical composition comprising the compound according to claim 4.
